(19) United States
(12) Patent Application Publication
(10) Pub. No.: US 2017/0015693 A1
(43) Pub. Date: Jan. 19, 2017

(54) ACRYLAMIDE-BASED CONDUCTIVE COMPOUNDS, AND METHODS OF PREPARATION AND USES THEREOF

(71) Applicant: EMPIRE TECHNOLOGY DEVELOPMENT LLC, Wilmington, DE (US)

(72) Inventors: William Brenden CARLSON, Seattle, WA (US); Gregory David PHELAN, Cortland, NY (US); Vincenzo CASASANTA, III, Woodinville, WA (US)

(73) Assignee: EMPIRE TECHNOLOGY DEVELOPMENT LLC, Wilmington, DE (US)

(21) Appl. No.: 15/123,985

(22) PCT Filed: Mar. 4, 2014
(86) PCT No.: PCT/US2014/020365

§ 371 Date: Sep. 6, 2016

(51) Int. Cl. C07H 5/04; C08F 220/58; H01M 10/0525; C08B 15/06

(52) U.S. Cl. CPC C07H 5/04; C08B 15/06; C08F 220/58; H01M 10/0525; H01M 2300/0082; H01M 2300/0085

(57) ABSTRACT

Acrylamide-based conductive compounds, and methods of making and using the acrylamide-based conductive compounds are disclosed. The acrylamide-based conductive compounds may include monomers or may be formed into polymers and acrylamide-based conductive materials, such as gels. The acrylamide-based conductive materials may be formed using inflammable or high boiling point fluids. The acrylamide-based conductive compounds described herein may conduct, coordinate, or otherwise be associated with various ions, including, without limitation, lithium ions, sodium ions and potassium ions. As such, acrylamide-based conductive compound may be used to support ion movement within electrical components and/or power devices such as batteries and capacitors.

### BACKGROUND

Use of lithium ion batteries as a power source for both consumer and industrial applications continues to expand. For example, lithium ion batteries are commonly used for consumer electronics devices, particularly mobile devices, as well as for battery-powered forklifts, automatic guided vehicles, and solar and wind power storage systems. The particular configuration of a lithium ion battery may depend on the device or equipment being powered by the battery. For instance, portable electronic devices often use electrodes formed from lithium cobalt oxide, which provides a high energy density and a slow loss of charge when the device is not in use. Industrial applications may be more likely to use electrodes formed from lithium iron phosphate or lithium nickel manganese cobalt oxide which have a lower energy density but generally provide a longer life and are safer than other forms of lithium ion batteries.

A typical lithium battery includes an anode and a cathode arranged in an electrolyte. Lithium ions move from the anode to the cathode to provide an electric current to power a device and may move back from the cathode to the anode to recharge the battery. The anode may be formed from a lithium-based material including a lithium salt and a counterion such as cobalt oxide. The various different counterions may provide differences in cell potential, energy storage and weight, generating two half-cell chemical reactions that may ultimately produce the electric current. The cathode may be formed from non-lithium materials, such as carbon or silicon. The movement of lithium ions occurs within an electrolyte. In a conventional lithium ion battery, the electrolyte is formed into a gel using various solvents. A typical electrolyte is polyvinylidene fluoride formed into a gel using ethylene carbonate, diethyl carbonate, or dimethyl carbonate.

Although useful and versatile, lithium ion batteries are prone to thermal runaway due to uncontrolled discharge. Thermal runaway may cause a battery to enter an unsafe condition because of the significant production of heat during uncontrolled discharge. For example, high temperatures within a lithium ion battery may cause the electrolyte to decompose, liberating unsafe compounds, such as hydrofluoric acid. In addition, organic solvents within the battery may boil, sending organic solvent vapor from the battery. The organic solvents often have undesirable properties, such as being highly flammable, which may lead to a fire within the battery at high temperatures. Accordingly, it would be beneficial to provide a lithium ion battery having an electrolyte formed using water and/or other non-flammable compounds to increase the safety of the battery.

### SUMMARY

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

Some embodiments provide compounds for use in electrolyte gels, the electrolyte gels, polymers of such compounds, gels comprising such compounds and/or polymers, gels, batteries comprising such compounds, polymers and gels, as well as methods of making and/or using each.

Some embodiments provide a compound of formula I:

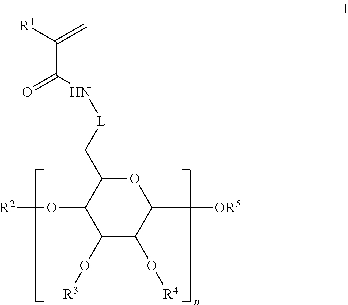

wherein L is a linking moiety selected from a direct bond, —O—, —R⁶—, —R⁶—O—, or —R⁶—O—C(O)—R⁶—; R¹ is H, —CH₃, or —C₁-C₆ alkyl-OH; R² is H or —CH₃; R³ is H or —CH₃; R⁴ is H or —CH₃; R⁵ is H or —CH₃; each R⁶ is independently selected from —O—, —R⁷—, —R⁷—O—, or —R⁷—O—C(O)—R⁷—; each R⁶ is independently selected from C₁-C₆ alkyl; and n is an integer of 1 to 100. In some embodiments, R¹, R², R³, R⁴, and/or R⁵ is independently selected from an alkyl group, including, without limitation, a butyl group, a propyl group, and a hexyl group. In some embodiments, R¹, R², R³, R⁴, and/or R⁵ is independently selected from an alkene group, an alkyne group, an aryl group, or an aromatic compound.

Some embodiments provide a polymer of formula II:

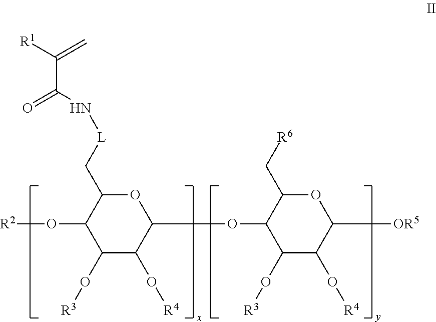

the polymer being a random polymer, a block polymer or an alternating polymer;

wherein L is a linking moiety selected from a direct bond, —O—, —R7-, —R7-O—, —R7-O—C(O)—R7-; R1 is H, —CH3, or —C1-C6 alkyl-OH; R2 is H or —CH3; R3 is H or —CH3; R4 is H or —CH3; R5 is H or —CH3; each R6 is independently —C1-C6 alkyl-OH; each R7 is independently selected from —O—, —R8-, —R8-O—, —R8-O—C(O)—R8-; each R8 is independently selected from C1-C6 alkyl; x is an integer of 1 to 100; and y is an integer of 0 to 100. In some embodiments, R1, R2, R3, R4, and/or R5 is independently selected from an alkyl group, including, without limitation, a butyl group, a propyl group, and a hexyl group. In some embodiments, R1, R2, R3, R4, and/or R5 is independently selected from an alkene group, an alkyne group, an aryl group, or an aromatic compound.

Some embodiments provide a conductive gel formed from a polymer comprising a structural unit derived from a compound of formula III:

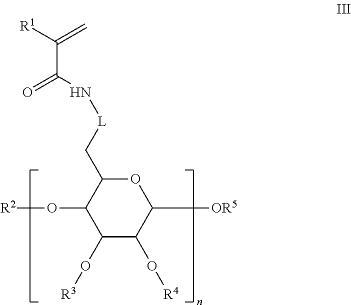

wherein L is a linking moiety selected from a direct bond, —O—, —R⁶—, —R⁶—O—, or —R⁶—O—C(O)—R⁶—; R² is H, —CH₃, or —C₁-C₆ alkyl-OH; R² is H or —CH₃; R³ is H or —CH₃; R⁴ is H or —CH₃; R⁵ is H or —CH₃; each R⁶ is independently selected from —O—, —R⁷—, —R⁷—O—, or —R⁷—O—C(O)—R⁷—; each R⁷ is independently selected from C₁-C₆ alkyl; and n is an integer of 1 to 100. In some embodiments, R¹, R², R³, R⁴, and/or R⁵ is independently selected from an alkyl group, including, without limitation, a butyl group, a propyl group, and a hexyl group. In some embodiments, R¹, R², R³, R⁴, and/or R⁵ is independently selected from an alkene group, an alkyne group, an aryl group, or an aromatic compound.

Some embodiments provide a method of making an acrylamide-based conductive material comprising contacting an acetone/pyridine solution with a carbonate solution to form a first intermediate compound and contacting 1-methyl-6-deoxy-6-ammonium bromide-D-glucose with the first intermediate compound to form a second intermediate compound. Hexane may be contacted with (meth) acryloyl chloride to form a third intermediate compound. A fourth intermediate compound may be formed by contacting the third intermediate compound with the second intermediate compound. Water may be removed from the fourth intermediate compound to form a solid compound. An alcohol may be contacted with the solid compound to dissolve organic material in the solid compound and subsequently removed.

Some embodiments provide an acrylamide-based conductive gel battery comprising an acrylamide-based conductive gel infused with electrolyte and at least one anode and at least one cathode arranged within the acrylamide-based conductive gel, wherein the acrylamide-based conductive gel supports ionic communication between the at least one anode and the at least one cathode, the ionic communication generating an electric current for the acrylamide-based conductive gel battery.

Some embodiments provide a compound of formula IV:

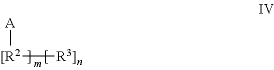

wherein A is an acrylamide moiety represented by the formula V:

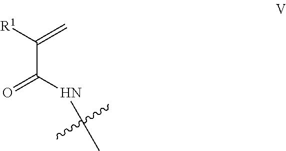

wherein R1 is H or —CH3; R2 is a polysaccharide, glucose, fructose, cellulose, hydroxyethyl cellulose, cellulose acetate butyrate, ethyl cellulose, a mogroside, mogroside II A1, mogroside II A2, mogroside II B, mogroside V, mogroside VI, and 7-oxomogroside II E; R3 is a polysaccharide, glucose, fructose, cellulose, hydroxyethyl cellulose, cellulose acetate butyrate, ethyl cellulose, a mogroside, mogroside II A1, mogroside II A2, mogroside II B, mogroside V, mogroside VI, and 7-oxomogroside II E; m is an integer of 1 to 100; and n is an integer of 0 to 100.

### BRIEF DESCRIPTION OF THE DRAWINGS

FIG. 1 depicts an illustrative synthesis pathway for an acrylamide-based conductive compound according to an embodiment.

FIG. 2 depicts an illustrative flow diagram for producing acrylamide-based conductive compounds according to an embodiment.

FIG. 3 depicts an illustrative reaction container configuration used in a method of producing acrylamide-based conductive compounds according to an embodiment.

FIG. 4 depicts an illustrative nuclear magnetic resonance (NMR) spectroscopy diagram of a first acrylamide-based conductive compound according to an embodiment.

FIG. 5 depicts an illustrative NMR spectroscopy diagram of a second acrylamide-based conductive compound according to an embodiment.

FIG. 6A depicts a non-limiting illustration of coordination of one lithium ion by an acrylamide-based conductive compound according to an embodiment.

FIG. 6B depicts a non-limiting illustration of coordination of two lithium ions by an acrylamide-based conductive compound according to an embodiment.

FIG. 7 depicts an illustrative mass spectrometry diagram of an acrylamide-based conductive compound coordinating at least one ion according to an embodiment.

FIG. 8 depicts an illustrative battery according to some embodiments.

FIG. 9 depicts the formation of an acrylamide-based conductive gel using hydroxyethyl cellulose (HEC) according to an embodiment.

### DETAILED DESCRIPTION

The terminology used in the description is for the purpose of describing the particular versions or embodiments only, and is not intended to limit the scope.

The described technology generally relates to acrylamide-based conductive compounds and methods for generating the acrylamide-based conductive compounds and forming the compounds into various configurations and/or materials, including polymers, gels or the like. According to some embodiments, the acrylamide-based conductive compounds may include an acrylamide moiety linked, bonded, or otherwise connected to a carbohydrate moiety, including, but not limited to, D-glucose. For example, an acrylamide-based conductive compound may be 1-methyl-6-deoxy-6-(meth)acrylamide-D-glucose, and can be synthesized according to some embodiments described herein.

According to some embodiments, the acrylamide-based conductive compounds may be formed into various acrylamide-based conductive materials, such as various gels. An acrylamide-based conductive compound and/or acrylamide-based conductive material may be incorporated into various devices, such as an electrical component and/or power device. Non-limiting examples of power devices include batteries and capacitors (for instance, an electrochemical double layer capacitor). For instance, an acrylamide-based conductive compound may be used within a battery as an anode and/or cathode binding material and/or as a gelation material for the battery electrolyte. An acrylamide-based conductive compound may be configured to conduct, coordinate, or otherwise be associated with various ions, including, without limitation, lithium ions, sodium ions and potassium ions. Accordingly, acrylamide-based conductive compounds and/or acrylamide-based conductive materials may be used to coordinate ions within a battery. For example, an acrylamide-based conductive gel may be used to coordinate lithium ions within a lithium ion battery.

In some embodiments, an acrylamide-based conductive compound may be of formula I:

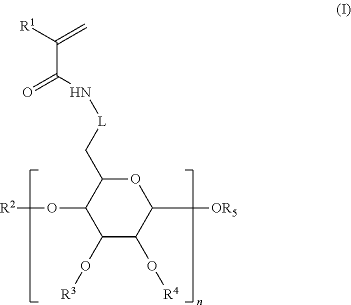

wherein L is a linking moiety selected from a direct bond, —O—, —R⁶—, —R⁶—O—, or —R⁶—O—C(O)—R⁶—;

each R¹ is H, —CH₃ or —C₁-C₆ alkyl-OH;

each R², R³, R⁴, and/or R⁵ is independently selected from H or —CH₃;

each R₆ is independently selected from —O—, —R⁷—, —R⁷—O—, or —R—O—C(O)—R⁷—;

each R⁷ is independently selected from C₁-C₆ alkyl; and

n is an integer of 1 to 100.

For example, n may be 1, 5, 10, 20, 50, 75, 100, or any value or range between any two of these values (including endpoints). In some embodiments, n may be 1 to 50. In some embodiments, n may be 50 to 100.

In some embodiments, each of R¹, R², R³, and R⁴ may be H. In some embodiments, each of R¹, R², R³, and R⁴ may be —CH₃. In some embodiments, R⁵ may be H. In some embodiments, R⁵ may be —CH₃. In some embodiments, R₂ may be —CH₃, R₃ may be H, and R⁴ may be H. In some embodiments, R⁶ may be —O—. In some embodiments, R₆ may be —R⁷—O—. In some embodiments, R₆ may be —R⁷—O—C(O)—R⁷—. In some embodiments, L may be a direct bond. In some embodiments, L may be —O—. In some embodiments, L may be —R⁶—O—C(O)—R⁶—. In some embodiments, R¹, R², R³, R⁴, and/or R⁵ is independently selected from an alkyl group, including, without limitation, a butyl group, a propyl group, and a hexyl group. In some embodiments, R¹, R², R³, R⁴, and/or R⁵ is independently selected from an alkene group, an alkyne group, an aryl group, or an aromatic compound.

Non-limiting examples of compounds represented by formula I include, but are not limited to, the following compounds:

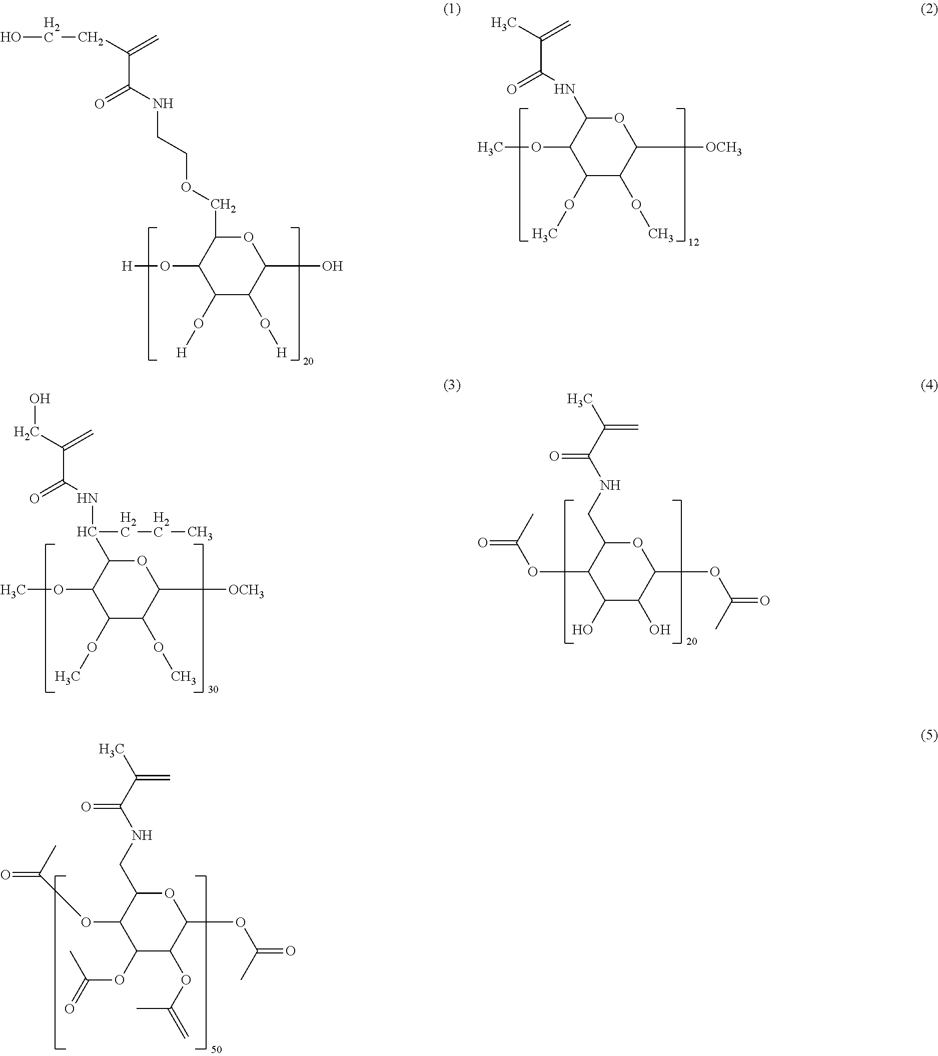

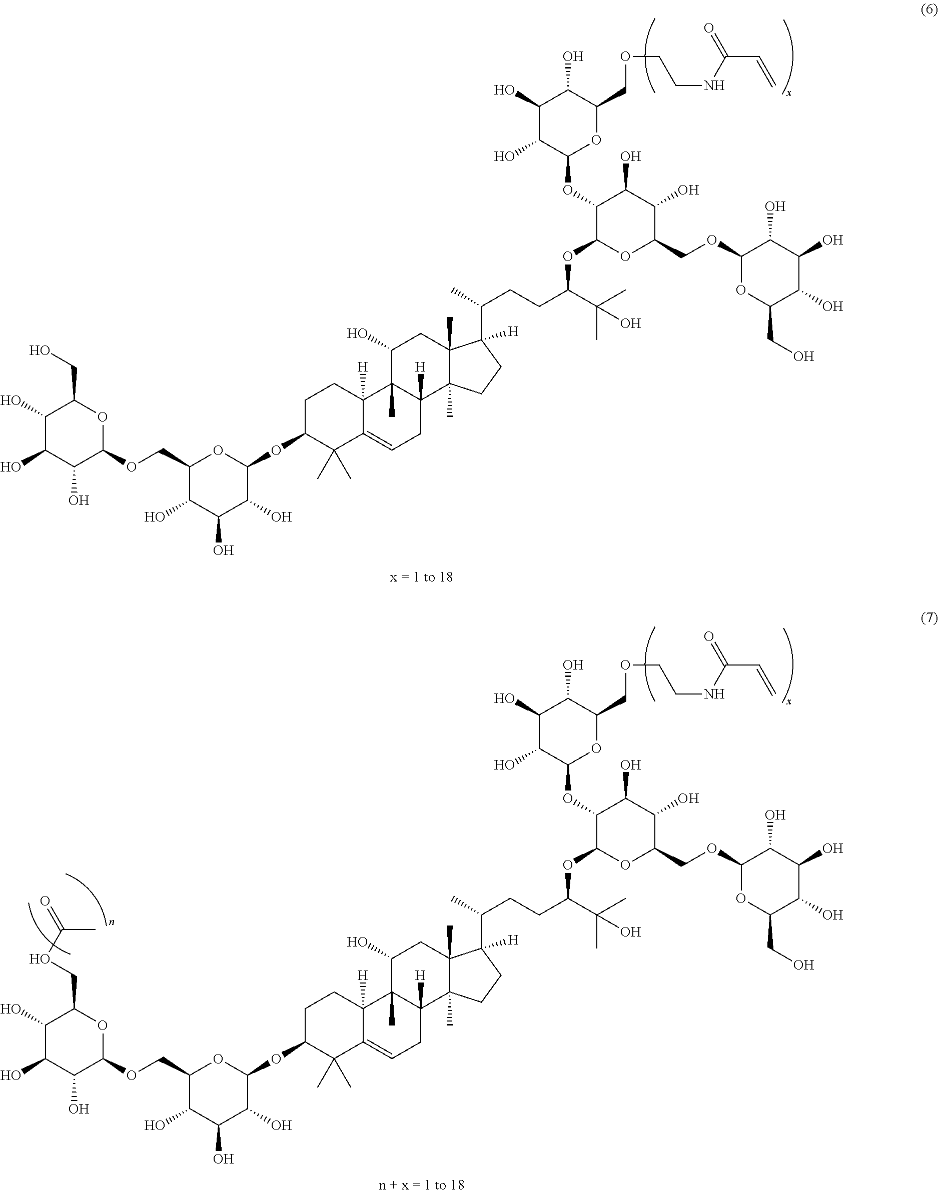

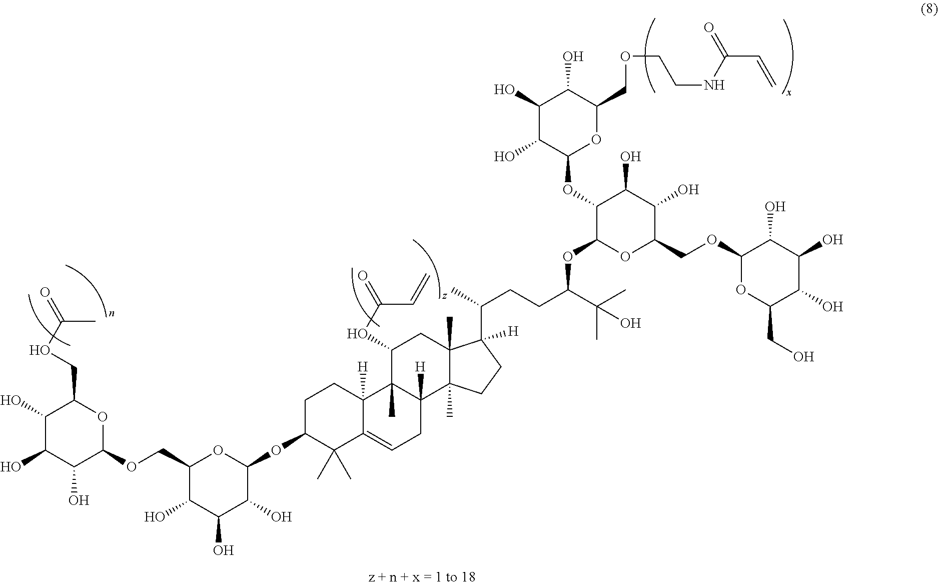

In some embodiments, an acrylamide-based conductive compound may be of formula II:

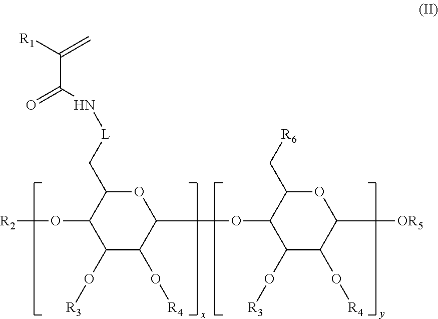

wherein the acrylamide-based conductive compound of formula II may be a random, block or alternating polymer;

L is a linking moiety selected from a direct bond, —R⁷—, —R⁷—O—, —R⁷—O—C(O)—R⁷—;

each R¹ is H, —CH₃, or —C¹—C₆ alkyl-OH;

each R², R³, R⁴, and/or R⁵ is H or —CH₃;

each R⁶ is independently —C₁-C₆ alkyl-OH;

each R⁷ is independently selected from —O—, —R⁸—, —R⁸—O—, —R⁸—O—C(O)—R⁸—;

each R⁸ is independently selected from C₁-C₆ alkyl;

x is an integer of 1 to 100; and

In some embodiments, y may be an integer of 1 to 100.

In some embodiments, L may be a direct bond. In some embodiments, L may be —O—. In some embodiments, L may be —R⁷—O—C(O)—R⁷—.

In some embodiments, R⁷ may be —O—. In some embodiments, R⁷ may be —R⁸—O—. In some embodiments, R⁷ may be —R⁸—O—C(O)—R⁸—. In some embodiments, R², R³, R⁴, and/or R⁵ may be H. In some embodiments, R², R³, R⁴, and/or R⁵ may be —CH₃. In some embodiments, R⁵ may be H. In some embodiment, R⁵ may be —CH₃. In some embodiments, R² may be —CH₃, R³ may be H and R⁴ may be H. In some embodiments, R², R³, R⁴, R⁵, and/or R⁶ is independently selected from an alkyl group, including, without limitation, a butyl group, a propyl group, and a hexyl group. In some embodiments, R², R³, R⁴, R⁵, and/or R⁶ is independently selected from an alkene group, an alkyne group, an aryl group, or an aromatic compound.

In some embodiments, x may be 1, 5, 10, 20, 25, 30, 40, 50, 75, 100, or any value or range between any two of these values (including endpoints). In some embodiments, y may be 1, 5, 10, 20, 25, 30, 40, 50, 75, 100, or any value or range between any two of these values (including endpoints). In some embodiments, x may be 1 to 50. In some embodiments, x may be 50 to 100. In some embodiments, y may be 1 to 50. In some embodiments, y may be 1 to 100.

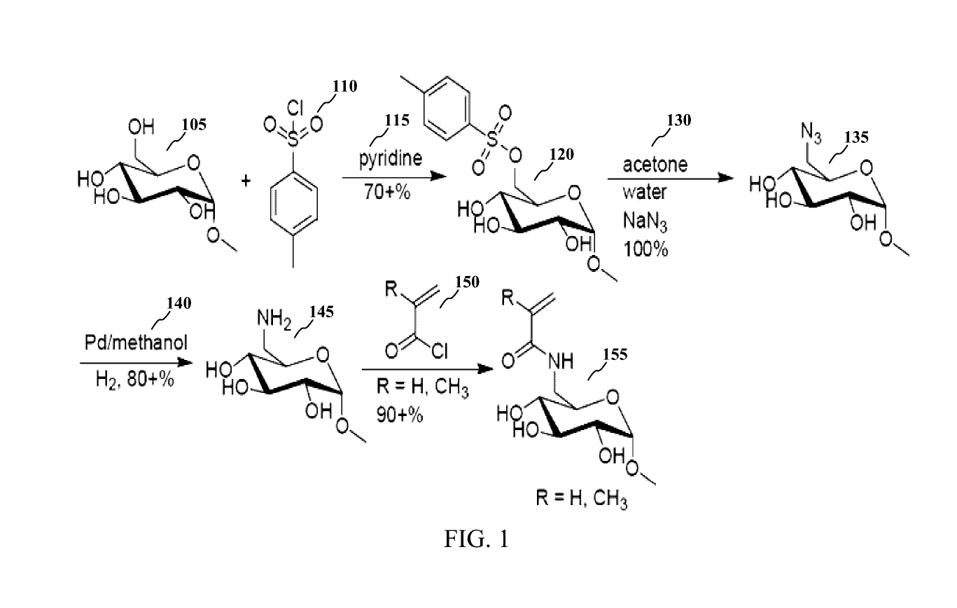

Acrylamide-based conductive compounds according to some embodiments may be formed using various methods and synthesis pathways. FIG. 1 depicts an illustrative synthesis pathway for an acrylamide-based conductive compound according to an embodiment. As shown in FIG. 1, glucose sugar 105 may be reacted with an acid chloride of p-toluene sulfonic acid 110 in pyridine 115. The adduct 120 may be reacted with sodium azide in water/acetone 130. The resultant azide 135 may be reduced using palladium on carbon 140 and the resulting amine 145 may be reacted with the acid chloride of (meth)acrylic acid 150 to form the acrylamide-based conductive compound 155.

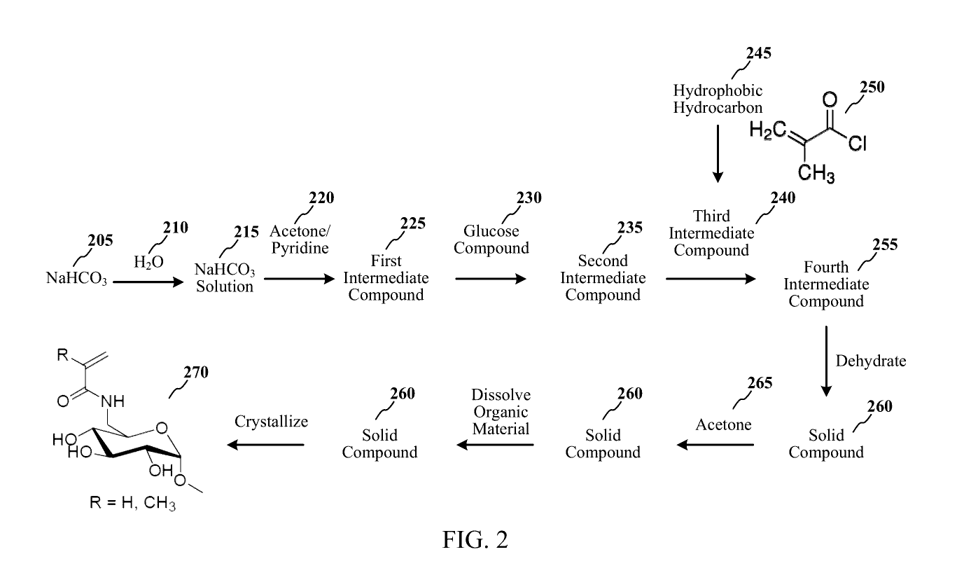

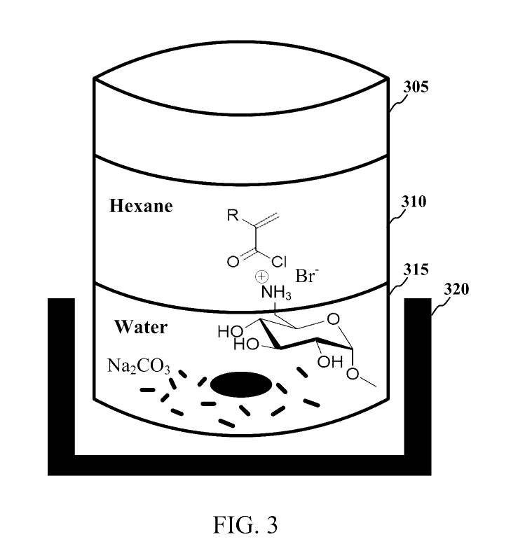

FIG. 2 depicts an illustrative flow diagram for producing acrylamide-based conductive compounds according to another embodiment. A carbonate solution 215 may be formed by dissolving a carbonate compound 205 in water 210, for example, in a first reaction container, such as the illustrative glass beaker depicted in FIG. 3 and described in more detail below. The carbonate compound 205 may include, without limitation, a carbonate, a bicarbonate, sodium carbonate, lithium carbonate, sodium bicarbonate, and/or lithium bicarbonate.

The carbonate compound 205 may be dissolved in the water 210, for instance, by stirring the water-sodium bicarbonate mixture for about one hour to about two hours. In an embodiment, once the carbonate compound 205 has dissolved in the water 210, the first reaction container may be placed in an ice bath and cooled, for instance, to about 0° C. to about 2° C. A first intermediate compound 225 may be formed by contacting an acetone/pyridine solution 220 with the carbonate solution 215. A second intermediate compound 235 may be formed by contacting a glucose compound 230 with the first intermediate compound 225. In an embodiment, the glucose compound 230 may include 1-methyl-6-deoxy-6-ammonium bromide-d-glucose. In an embodiment, the glucose compound 230 may be dissolved by stirring. In some embodiments, the ammonium bromide component may include, without limitation, ammonium chloride, bromide, iodide, or tosylate.

A third intermediate compound 240 may be formed by contacting a hydrophobic hydrocarbon 245 with (meth) acryloyl chloride 250. In some embodiments, the hydrophobic hydrocarbon 245 may include hexane. A fourth intermediate compound 255 may be formed by contacting the third intermediate compound 240 with the second intermediate compound 235, for example, in the first reaction container. The third intermediate compound 240 may be added drop-wise to the second intermediate compound 235 in the first reaction container such that the third intermediate compound forms a layer on (the water of) the second intermediate compound (for instance, see FIG. 3). The fourth intermediate compound 255 may be stirred, for example, for about 6 hours, about 12 hours, about 24 hours or values or ranges between these values (including endpoints).

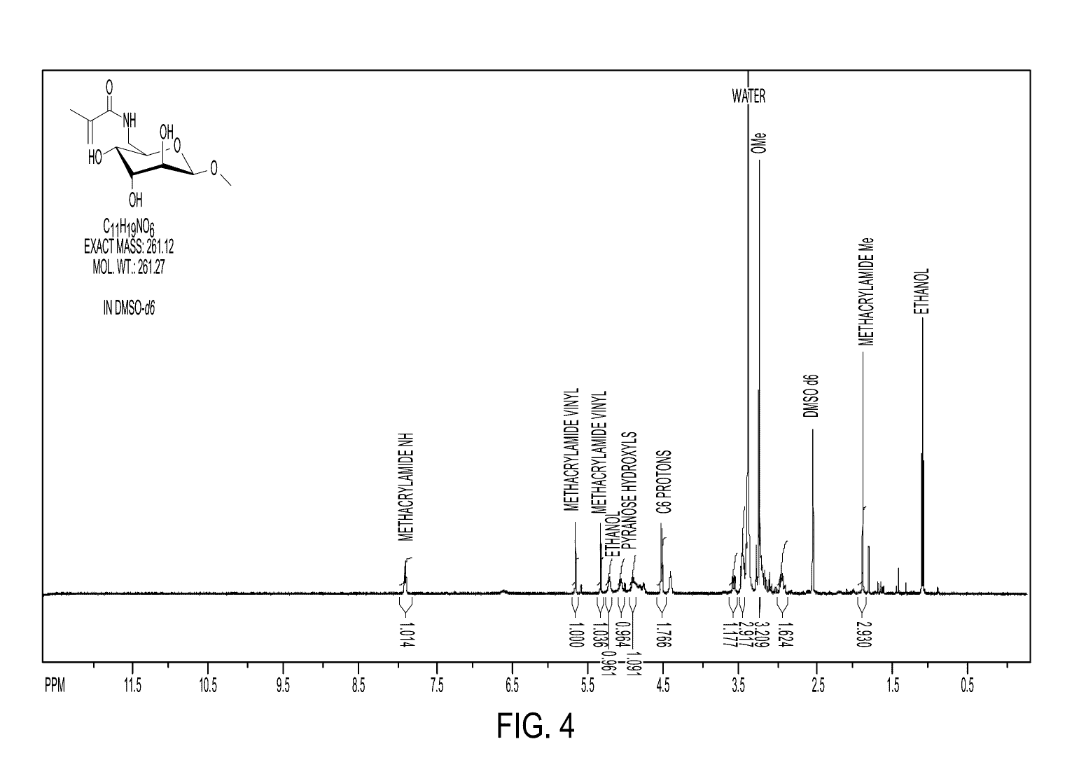

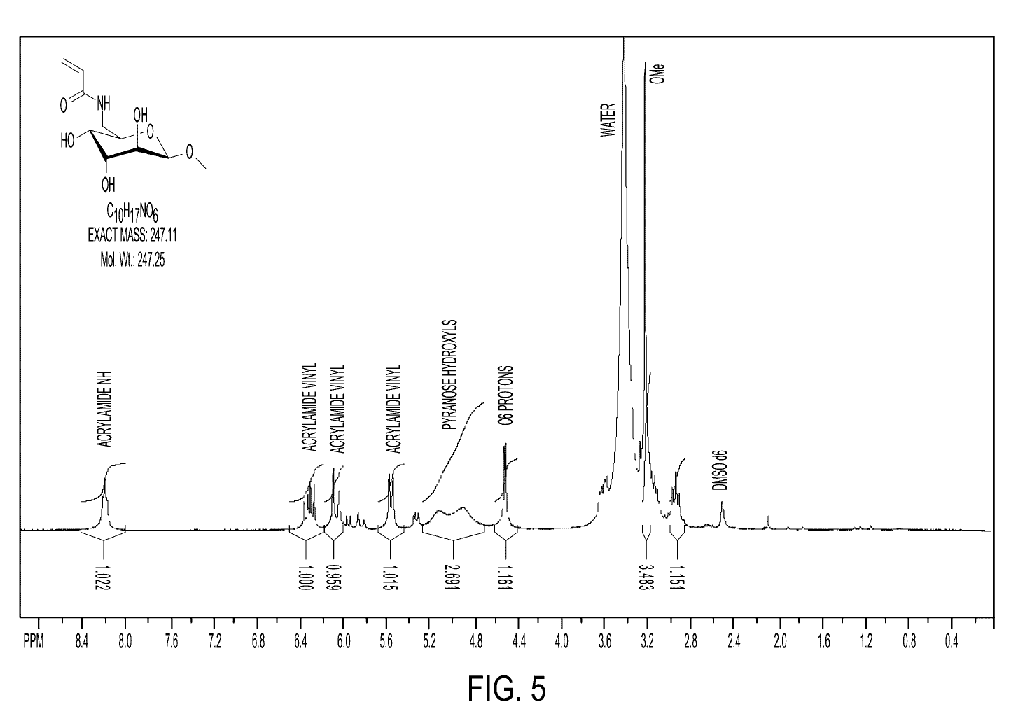

A solid compound 260 may be generated by removing water from (dehydrating) the fourth intermediate compound 255. For example, the contents of the first reaction container (in other words, the fourth intermediate compound 255) may be poured into a separatory funnel and the water may be removed to a third container. In an embodiment, the water may be removed using rotary evaporation. Acetone 265 may be contacted with the solid compound 260 and the acetone evaporated. For example, the acetone 265 may be evaporated using rotary evaporation. The organic materials in the solid compound 260 may be dissolved. For instance, ethanol may be added to the solid compound 260 to dissolve the organic materials while the inorganic materials are not dissolved. The ethanol may be filtered and removed using rotary evaporation. The acrylamide-based conductive compounds 270 may be formed by allowing the solid compound to crystallize or recrystallize, for example, using methanol/acetone. Illustrative and non-restrictive examples of acrylamide-based conductive compounds 270 (for instance, 1-methyl-6-deoxy-6-(meth)acrylamide-D-glucose) formed through the method described in FIG. 2 are depicted in the nuclear magnetic resonance (NMR) spectroscopy diagrams depicted in FIGS. 4 and 5.

An acrylamide-based conductive compound formed through the methods described in FIG. 1 and/or FIG. 2 may generate a compound in which the acrylamide moiety is located at C₆, as shown in formulas I and II above. However, embodiments are not so limited, as the acrylamide moiety may be located at other positions of the ring structure, including C₁-C₄. For example, an azo intermediate may be formed that is reduced to an amine, for instance, obtained through hydrolysis of the amide. The acrylamide may be obtained from the amino sugar. Acrylamides may be obtained through reactions with an acid chloride, acid anhydride, or though the ammonium salt. The ammonium salt may be obtained through a reaction between an amide and an acid. In addition, acrylamide may be obtained by reacting (meth)acrylic acid with an amine and then heating the product under conditions (for example 130° C.) effective to remove water and to obtain the acrylamide.

FIG. 3 depicts an illustrative reaction container configuration for the method of producing acrylamide-based conductive compounds depicted in FIG. 2 according to an embodiment. As shown in FIG. 3, a reaction container (for instance, the first reaction container) 305 may be positioned within a coolant 320, such as an ice bath. The contents of the reaction container 305 as depicted in FIG. 3 may be the fourth intermediate compound 255 formed by contacting the third intermediate compound 240 with the second intermediate compound 235. For example, the reaction container 305 may hold, before mixing, a layer 310 including the third intermediate compound 240 on a layer 315 that includes the second intermediate compound 235.

In some embodiments, a conductive gel may include a polymer having a structural unit derived from a compound represented by the formula III:

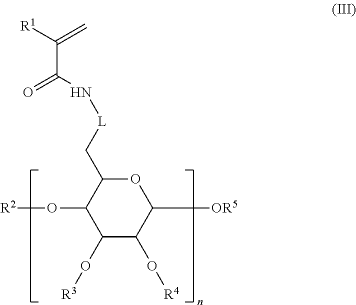

wherein L is a linking moiety selected from a direct bond, —R⁶—, —R⁶—O—, or —R⁶—O—C(O)—R⁶—;

each R¹ is H, —CH₃ or —C₁-C₆ alkyl-OH;

each R², R³, R⁴, and/or R⁵ is H or —CH₃;

each R⁶ is independently selected from —O—, —R⁷—, —R⁷—O—, or —R—O—C(O)—R⁷—,

each R⁷ is independently selected from C₁-C₆ alkyl; and

n may be an integer of 1 to 3000.

In some embodiments, each of R¹, R², R³, and R⁴ may be H. In some embodiments, each of R¹, R², R³, and R⁴ may be —CH₃. In some embodiments, R⁵ may be H. In some embodiments, R⁵ may be —CH₃. In some embodiments, R² may be —CH₃, R³ may be H, and R⁴ may be H. In some embodiments, R⁶ may be —O—. In some embodiments, R⁶ may be —R⁷—O—. In some embodiments, R⁶ may be —R—O—C(O)—R⁷—. In some embodiments, L may be a direct bond. In some embodiments, L may be —O—. In some embodiments, L may be —R⁶—O—C(O)—R⁶—. In some embodiments, each of R¹, R², R³, and R⁴ may be an alkyl group, including, without limitation, a butyl group, a propyl group, and a hexyl group. In some embodiments R¹, R², R³, and R⁴ is independently selected from an alkene group, an alkyne group, an aryl group, or an aromatic compound

In some embodiments, n may be 1, 100, 200, 500, 750, 1000, 1500, 2000, 2500, 3000, or any value or range between any two of these values (including endpoints). In some embodiments, n may be 1 to 1500. In some embodiments, n may be 1500 to 3000.

Although the acrylamide moiety is depicted in formulas I and II as being at the sixth position at C₆, some embodiments are not so limited, as the acrylamide moiety may be located at any of the positions of the ring structure, including C₁-C₄ with appropriate shifting of the remaining ring constituents.

According to some embodiments, acrylamide-based conductive compounds may be formed into polymers. In an embodiment, monomers of the acrylamide-based conductive compound may be polymerized by chain growth techniques. In another embodiment, monomers of the acrylamide-based conductive compound may be polymerized using a crosslinking agent. The following formula VI is an illustrative and non-restrictive crosslinking agent linking acrylamide moieties of two acrylamide-based conductive compound monomers:

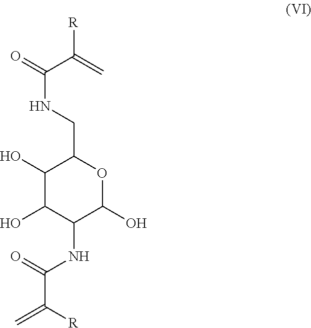

In an embodiment, the acrylamide-based conductive component monomer (for example, 1-methyl-6-deoxy-6(meth)acrylamide-D-glucose) may be dissolved into a solvent, such as glycerol, to form an acrylamide-based conductive component solution. A crosslinking agent may be added to the acrylamide-based conductive component solution. In an embodiment, the crosslinking agent may include 2-deoxy-6-deoxy-2-(meth)acrylamide-6-(meth)acrylamide-D-glucose and an initiator. For example, an initiator may include azobisisobutyronitrile (MEW) or a metal persulfate, such as lithium persulfate. The acrylamide-based conductive component solution and crosslinking agent may be heated, for instance, to about 70° C. to about 80° C. to form an acrylamide-based conductive gel. In some embodiments, the acrylamide-based conductive component solution and crosslinking agent may be heated to about 70° C., about 72° C., about 74° C., about 76° C., about 78° C., about 80° C., or any value or range between any two of these values (including endpoints).

As described above, some embodiments provide that an acrylamide-based conductive compound may be formed into a gel material. A non-limiting example provides that 1-methyl-6-deoxy-6 (meth)acrylamide-D-glucose may be synthesized as a gel material that may coordinate metal ions and, as such, may be used in metal ion batteries, such as lithium ion batteries. Acrylamide-based conductive compounds may be formed into a gel using various fluids. An illustrative fluid is diethyl carbonate. However, acrylamide-based conductive compounds may be formed into a gel using fluids that are inflammable or significantly less flammable than typical materials used to form materials in conventional metal ion batteries. For instance, very high boiling organic liquids can be used to form the acrylamide-based conductive materials, including, without limitation water, glycerol, sorbitol, sorbitol, ethylene glycol, dipropyl carbonate, propylene carbonate, cyclopentanone, cyclohexanone, and/or propylene glycol

In some embodiments, an acrylamide-based conductive compound may be of formula IV:

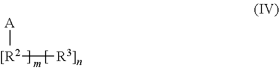

wherein A is an acrylamide moiety represented by the formula V:

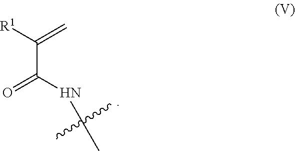

In some embodiments, R² may be a polysaccharide, glucose, fructose, cellulose, hydroxyethyl cellulose, cellulose acetate butyrate, ethyl cellulose, a mogroside, mogroside II A₁, mogroside II A₂, mogroside II B, mogroside V, mogroside VI, and 7-oxomogroside II E.

In some embodiments, R³ may be a polysaccharide, glucose, fructose, cellulose, hydroxyethyl cellulose, cellulose acetate butyrate, ethyl cellulose, a mogroside, mogroside II A₁, mogroside II A₂, mogroside II B, mogroside V, mogroside VI, and 7-oxomogroside II E

In some embodiments, R¹ may be H or —CH₃.

In some embodiments, R² and/or R³ may be glucose. In some embodiments, R² and/or R³ may be glucose or fructose. In some embodiments, R² and/or R³ may be cellulose. In some embodiments, R² and/or R³ may be hydroxyethyl cellulose, cellulose acetate butyrate, or ethyl cellulose. In some embodiments, R² and/or R³ may be a mogroside. Non-limiting examples of mogrosides include mogroside II A₁, mogroside II A₂, mogroside II B, mogroside V, mogroside VI, and 7-oxomogroside II E.

In some embodiments, m may be an integer of 1 to 3000. For example, m may be 1, 50, 100, 200, 500, 750, 1000, 1500, 2000, 2500, 3000, or any value or range between any two of these values (including endpoints). In some embodiments, n may be an integer of 1 to 3000. For example, n may be 1, 50, 100, 200, 500, 750, 1000, 1500, 2000, 2500, 3000, or any value or range between any two of these values (including endpoints). In some embodiments, n may be 1 to 50. In some embodiments, n may be 50 to 100. In some embodiments, m may be 0. In some embodiments, m may be 1 to 50.

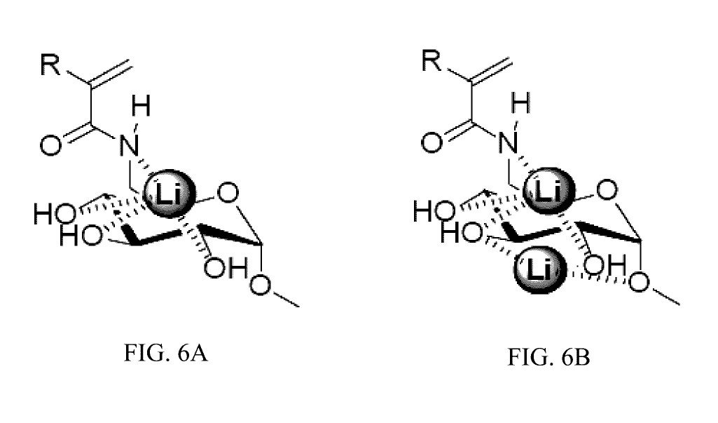

The acrylamide-based conductive compounds described according to some embodiments may coordinate one or more ions. In an embodiment, the acrylamide-based conductive compounds may coordinate metal ions, including, without limitation, ions of lithium, sodium, potassium, magnesium, cesium, calcium, rubidium, iron and/or copper. In an embodiment, the acrylamide-based conductive compounds may coordinate one or two ions. FIGS. 6A and 6B depict non-limiting illustrations of coordination of one lithium ion and two lithium ions, respectively, by an acrylamide-based conductive compound according to some embodiments. For example, the oxygens of the ring structure of the acrylamide-based conductive compound may have the ability to attract the positive charge of the cation. The acrylamide monomers and polymers formed therefrom are not ionic. The lone pairs on the nitrogen, oxygen and/or carbonyl may be capable of attracting the metal ions, for example, through electrostatic interactions. The acrylamide-based conductive compounds according to some embodiments are not bound to coordinate ions according to the example coordination configurations described herein (for example, the coordination configurations depicted in FIGS. 6A and 6B) as these are provided for illustrative purposes only. The acrylamide-based conductive compounds may coordinate ions according to any configuration capable of providing coordination according to some embodiments described herein.

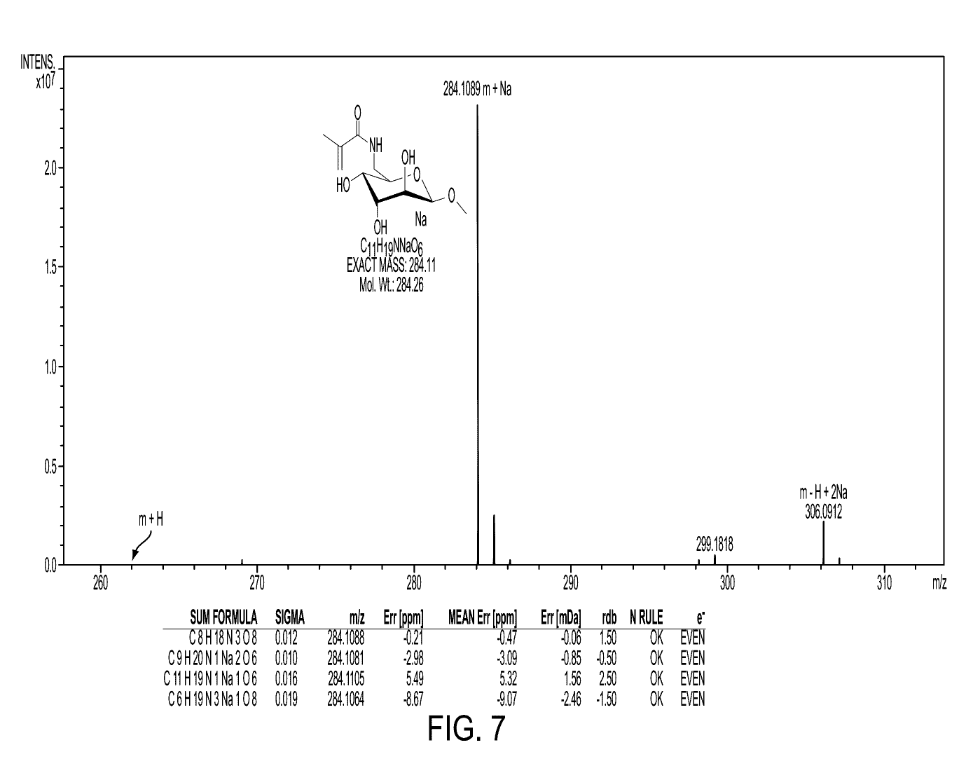

FIG. 7 depicts an illustrative mass spectrometry diagram of an acrylamide-based conductive compound coordinating at least one ion according to an embodiment. For example, the NMR spectroscopy diagram of FIG. 7 may depict 1-methyl-6-deoxy-6-(meth)acrylamide-D-glucose coordinating one or two sodium ions. In some embodiments, the metal ion may be loosely bound and able to migrate. In contrast, charged polymers may prevent the migration of coordinated cations. Accordingly, acrylamide-based conductive compounds and polymers and/or materials formed therefrom may be configured for applications requiring the migration of coordinated cations, such as a battery in which migration of a metal ion provides an electrical current.

According to some embodiments, an acrylamide-based conductive compound may be formed into various materials (acrylamide-based conductive materials) that may be used in battery applications. For instance, an acrylamide-based conductive compound may be formed into a gel material. A non-limiting example provides that 1-methyl-6-deoxy-6 (meth)acrylamide-D-glucose may be synthesized as a gel material that may coordinate metal ions and, as such, may be used in metal ion batteries, such as lithium ion batteries. Acrylamide-based conductive compounds may be formed into a gel using various fluids. An illustrative fluid is diethyl carbonate. However, acrylamide-based conductive compounds may be formed into a gel using fluids that are inflammable or significantly less flammable than typical materials used to form materials in conventional metal ion batteries. For instance, very high boiling organic liquids can be used to form the acrylamide-based conductive materials, including, without limitation water, glycerol, sorbitol, sorbitol, ethylene glycol, dipropyl carbonate, propylene carbonate, cyclopentanone, cyclohexanone, or propylene glycol. In addition, acrylamide-based conductive materials formed using these organic liquids are non-toxic, environmentally benign, and biodegrade in the environment under either aerobic or anaerobic conditions. In an embodiment, such acrylamide-based conductive materials may be used with electrolyte solutions of lithium compounds to create the gels.

Acrylamide-based conductive materials according to some embodiments may be formed using a wide variety of organic liquids that are non-flammable and/or have very high boiling points generating a material that may coordinate metal ions and make them more freely available for energy producing purposes. For example, the coordination of ions by the acrylamide-based conductive materials may solvate the ions using the polymer binder itself. This may prevent or reduce runaway reactions and spontaneous decomposition of the battery by preventing very fast diffusion of the ions, while allowing for effective and efficient performance of the battery. For instance, glycerol has a very high boiling point (about 290° C.) and, as such, glycerol may remain in the liquid state during a thermal runaway event. In another instance, if water is used as the solvent then steam may be given off during a thermal runaway event which is not toxic or explosive.

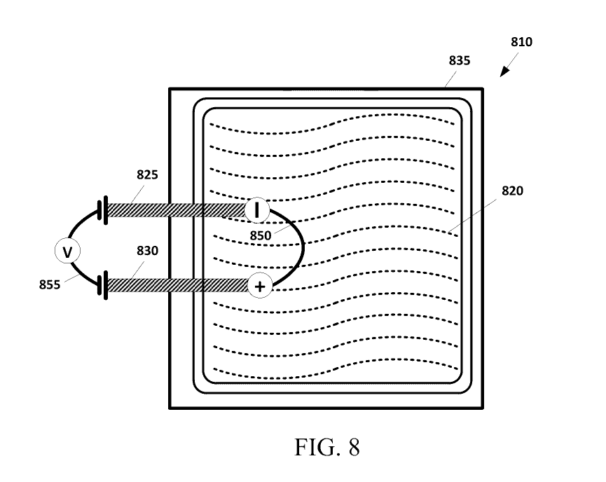

FIG. 8 depicts an illustrative battery according to some embodiments. As shown in FIG. 8, a battery 810 may include a cathode 825 and an anode 830 in contact with electrolyte 820 formed using acrylamide-based conductive materials according to some embodiments. The battery 810 may be configured as a metal ion battery, such as a lithium ion battery, having a case 835 configured to enclose the electrolyte 820, the anode 830 and the cathode 825. The acrylamide-based conductive materials may coordinate the metal ions such that the electrolyte 820 supports a flow of ions 850 between the anode 830 and the cathode 825. The flow of ions 850 between the anode 830 and the cathode 825 may operate to generate a voltage 855 for the battery 810. For example, the voltage 855 may be at least about 0.9 Volts. In a further example, the voltage 855 may be about 0.9 Volts to about 4.2 Volts. In some embodiments, the voltage may be about 0.9 Volts, about 2.0 Volts, about 3.5 Volts, about 3.0 Volts, about 3.5 Volts, about 4.0 Volts, about 4.2 Volts or any value or range between any two of these values (including endpoints). In an embodiment, the acrylamide-based conductive materials may also be configured as a binder for the anode 830 and the cathode 825. In an embodiment, the battery 810 may operate as a power supply to one or more electrical devices, circuits, or the like in electrical connection with the battery.

The anode 830 may include various lithium compounds. For example, the anode 830 (for example, the negative electrode) may include any lithium host material that can sufficiently undergo lithium intercalation and de-intercalation while functioning as the negative terminal of the lithium ion battery. In some embodiments, the negative electrode may also include a polymer binder material to structurally hold the lithium host material together. For instance, the anode 830 may be formed from graphite in combination with at least one of polyvinylidene fluoride (PVDF), an ethylene propylene diene monomer (EPDM) rubber, carboxymethyl cellulose (CMC), sugar and/or carbohydrate derivatives, and/or combinations thereof. In some embodiments, graphite may be used to form the anode 830 because, among other things, graphite exhibits favorable lithium intercalation and de-intercalation characteristics, is relatively non-reactive, and can store lithium in quantities that produce a relatively high energy density. Non-limiting forms of graphite include graphite produced by Timcal Graphite & Carbon of Bodio, Switzerland, Lonza Group of Basel, Switzerland, or Superior Graphite of Chicago, Ill., United States. The anode 830 may include other materials such as lithium titanate. In some embodiments, the negative-side current collector of the anode 830 may be formed from copper or any other appropriate electrically conductive material known to those having ordinary skill in the art.

The cathode 825 (for example, the positive electrode) may be formed from various lithium-based active materials. For example, the cathode 825 may be formed from any lithium-based active material that can sufficiently undergo lithium intercalation and de-intercalation while functioning as the positive terminal of the lithium ion battery. In some embodiments, the cathode 825 may also include a polymer binder material to structurally hold the lithium-based active material together. One class of known materials that can be used to form the cathode 825 is layered lithium transitional metal oxides. For example the cathode 825 may include at least one of spinel lithium manganese oxide (LiMn2O₄), lithium cobalt oxide (LiCoO₂), nickel-manganese-cobalt oxide [Li(NiₓMn_{y}CO_{z})O₂], lithium iron polyanion oxide, such as lithium iron phosphate (LiFePO₄) or lithium iron fluorophosphate (Li₂FePO₄F) in combination with at least one of polyvinylidene fluoride (PVDF), ethylene propylene diene monomer (EPDM) rubber, carboxymethyl cellulose (CMC), sugar or carbohydrate derivatives, and/or combinations thereof. Other lithium-based active materials may also be used to form the cathode 825, including, without limitation, lithium manganese phosphate, lithium vanadium phosphate, binary combinations of lithium iron phosphate, lithium manganese phosphate, or lithium vanadium phosphate, a lithiated binary oxide of two elements chosen from manganese, nickel, and cobalt, a lithiated ternary oxide of manganese, nickel, and cobalt, lithium nickel oxide (LiNiO₂), lithium aluminum manganese oxide (LiₓAl_{y}Mn_{1-y}O₂), and lithium vanadium oxide (LiV₂O₅), and/or combinations thereof. In some embodiments, the positive-side current collector of the anode 825 may be formed from aluminum or any other appropriate electrically conductive material known to those having ordinary skill in the art.

Non-polar, aprotic organic solvents have traditionally been used to create the gel materials used in metal ion batteries, such as lithium ion batteries. These solvents have typically been carbonates such as dimethyl carbonate. However, these solvents may lead to dangerous conditions within metal ion batteries, for example, during thermal runaway as described above. The dangerous conditions may lead to batteries combusting and/or exploding. Acrylamide-based conductive materials according to some embodiments may use solvents such as water, glycerol, sorbitol, sorbitol, ethylene glycol, dipropyl carbonate, propylene carbonate, cyclopentanone, cyclohexanone, propylene glycol, acetone, and/or ethanol that are inflammable or significantly less flammable than traditional solvents as these liquids are simple sugars or carbohydrates. The solvents can also be mixed together. The solvents together with other lithium compounds may form the electrolyte 820 that that is a solution of the lithium compounds in the glycerol, sorbitol, propylene glycol or some other solvent. These non-aqueous electrolytes 820 may use non-coordinating anion salts such as lithium hexafluorophosphate (LiPF₆), lithium hexafluoroarsenate monohydrate (LiAsF₆), lithium perchlorate (LiClO₄), lithium tetrafluoroborate (LiBF₄), and lithium triflate (LiCF₃SO₃).

In an embodiment, the acrylamide-based conductive materials may be configured as binders that hold the anode 830 and the cathode 825 together. In an embodiment, the acrylamide-based conductive materials may be configured as the gelation material for the electrolyte 820. In an embodiment in which the battery is configured as a lithium ion battery, the acrylamide-based conductive materials may be configured as the gelation material for the solution of LiPF₆, LiAsF₆, LiBF₄, LiClO₄, or LiCF₃SO₃.

In some embodiments, an acrylamide-based conductive gel may form the electrolyte 820 conductive medium between the anode 830 and the cathode 825 electrodes. In some embodiments, the electrolyte may include at least one of lithium hexafluorophosphate, lithium hexafluoroarsenate monohydrate, lithium perchlorate, lithium tetrafluoroborate, and lithium triflate. According to some embodiments, the anode 830 and the cathode 825 may be formed from slurries created from the dispersion of particulate materials of the electrodes in a polymer binder. Various forms of the acrylamide-based conductive compounds and/or materials may be used to create these slurries, including, for example, 1-methyl-6-deoxy-6-(meth)acrylamide-D-glucose; 1,2,3,4-tetramethyl-6-deoxy-6-acrylamide-D-glucose and/or 1-methyl-6-deoxy-6-acrylamide-D-glucose. In some embodiments, a thick solution of monomer in solvent may be formed, for example, in which the monomer and crosslinking agent content is greater than about 50%. Crosslinkers can be used in a variety of ways. One way is when they are used at less than 1% by mol. At this concentration they cause branching. Branching increases chain entanglement and an increase in viscosity but the system retains the ability to flow. As the concentration of crosslinker increases the result is a thermosetting system which then completely gels into a non-flowable media. The preferred concentration of crosslinker is between 0.5 and 5%. The compound used for the anode 830 and/or the cathode 825 may be dispersed into independent volumes of the acrylamide-based conductive compounds monomer-solvent solution to form a slurry. The acrylamide-based conductive compound monomer may then be polymerized and the solvent may be evaporated to form a solid or semi-solid compound of the electrode material.

In some embodiments, a crosslinking agent may be used at less than 1% by mole, for instance, to facilitate branching within the acrylamide-based conductive gel. Branching may increase chain entanglement and may facilitate an increase in viscosity while retaining an ability to flow. As the concentration of crosslinker increases beyond 1.5 to 2%, the acrylamide-based conductive gel may result is a thermosetting system which then completely gels into a non-flowable or essentially non-flowable media.

Although lithium ion batteries have been used as an example herein, embodiments are not so limited. The acrylamide-based conductive compounds, materials, gels, or the like may be used in any type of battery capable of operating according to some embodiments described herein, including, without limitation, lithium-nickel batteries, nickel hydroxide/metal hydride batteries, and batteries using metal ions such as sodium, potassium, magnesium, calcium, rubidium, cesium, iron and/or copper.

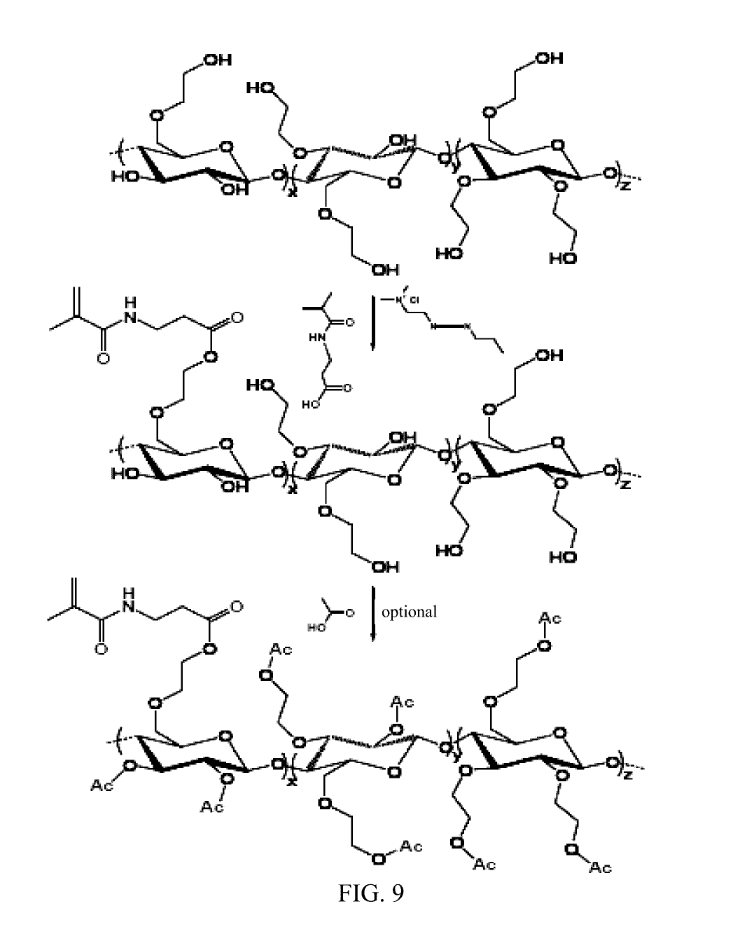

As described herein, various carbohydrate moieties may be used to form the acrylamide-based conductive compounds according to some embodiments. In an embodiment, derivatives of cellulose may be used to form the acrylamide-based conductive compounds. A non-limiting example of a cellulose derivative is hydroxyethyl cellulose (HEC). HEC may be functionalized with acrylamides for crosslinking and polymerization. The pyranose rings of HEC may coordinate lithium ions. HEC may be used for water or glycerol infused gels. In an embodiment, the hydroxy units of HEC may be capped with acetic acid moieties to form an organic solvent gel in which carbonate solvents can be used to infuse the gel. FIG. 9 depicts the formation of an acrylamide-based conductive gel using HEC according to an embodiment.

According to some embodiments, compounds such as cellulose acetate butyrate, ethyl cellulose, and amylose may also be used to form acrylamide-based conductive compounds and/or acrylamide-based conductive materials. In some embodiments, mogrosides may also be used to form acrylamide-based conductive compounds and/or acrylamide-based conductive materials. In general, mogrosides are highly branched molecules with five or more glucose units radiating off of a central steroid unit. Viscosity of acrylamide-based conductive materials using mogrosides may increase rapidly because of the branched nature of the mogroside molecules that facilitates the formation of gels. Acrylamide-based conductive materials using mogrosides may be polymerized or extended through reaction of the first carbon hydroxyls resulting in an even more highly branched system. The hydroxyl moieties can also capped with methacrylate or acrylamide moieties for chain extension polymerization and crosslinking. Mogrosides modified by this method may results in an acrylamide-based conductive gel that may be hydrated with water or glycerol. The hydroxyl moieties can be capped with acetyl or longer units along with the acrylamide or methacrylate derivative to form an acrylamide-based conductive gel that may be hydrated with organic solvents such as various carbonate solvents.

### EXAMPLES

### Example 1

### Preparation of an Acrylamide-Based Conductive Compound

An acrylamide-based conductive compound of 1-methyl-6-deoxy-6-(meth)acrylamide-D-glucose is formed using the following process.

About 11.5 grams of lithium carbonate is dispersed in about 100 milliliters of deionized water in a 1 liter glass beaker. The lithium carbonate dispersion is stirred for about 1.5 hours. The 1 liter glass beaker will be placed in an ice bath at a temperature of about 2° C. Not all the lithium carbonates dissolves. About 12 grams of 1-methyl-6-deoxy-6-ammonium bromide-d-glucose will be added to the 1 liter glass beaker and dissolved by stirring for about 4 hours.

About 100 milliliters of hexane and about 3.5 milliliters of (meth) acryloyl chloride will be added to a 250 milliliter glass beaker and stirred to make uniform. The contents of the 250 milliliter glass beaker will be added to the 1 liter glass beaker drop-wise. The contents of the 1 liter glass beaker will be stirred for about 12 hours. The contents of the 1 liter glass beaker will be poured into a separatory funnel to separate the water from hexanes. Rotary evaporation will also be used to remove water from the solid compound. The solid compound will be exposed to acetone, which will be removed through rotary evaporation. The solid compound will be exposed to ethanol to dissolve organic compounds. The dissolved organic compounds will be filtered from the solid compound and the ethanol removed through rotary evaporation and filtrations. The solid compound will be crystallized from acetone/ethanol to form 1-methyl-6-deoxy-6-(meth)acrylamide-D-glucose. The amino moieties discussed in this application appear primarily at the 6 position of the carbohydrate hexose ring. However, it is possible to have the amino at the 1, 2, 3, and 4 positions of the ring. It would be obvious to one skilled in the art to produce similar compounds with the amino and/or acrylamide moiety in another position of the sugar compound or use differing carbohydrates or sugar compounds.

### Example 2

### Preparation of an Acrylamide-Based Conductive Compound Using an Ammonium Sugar Intermediate

An acrylamide-based conductive compound of 1-methyl-6-deoxy-6-(meth)acrylamide-D-fructose will be formed.

The amide of an N-acetyl fructose compound will be hydrolyzed to form an amino fructose compound. The amino fructose compound will be reacted with (meth)acrylic acid to form an ammonium fructose (meth)acrylate compound (an “ammonium sugar” compound). Amide bond formation may be initiated by heating the ammonium sugar compound to about 100° C. to about 130° C. to remove water from the ammonium sugar compound. The dehydrated ammonium sugar compound may be crystallized to form the fructose acrylamide-based conductive compound

### Example 3

### Energy Density of an Acrylamide-Based Conductive Material

A monomer of an acrylamide-based conductive compound 1-methyl-6-deoxy-6-(meth)acrylamide-D-glucose will be formed with a molecular weight of about 284.26 grams/mole. The acrylamide-based conductive compound monomer will be used to form a gel material by mixing the acrylamide-based conductive compound monomer with crosslinking agents 2-deoxy-6-deoxy-2-(meth)acrylamide-6-(meth)acrylamide-D-glucose and lithium persulfate and heating the mixture to about 75° C. for about 2 hours. The gel material will be used as the electrolytic storage/transport medium within a lithium ion electrochemical double-layer capacitor.

The acrylamide-based conductive compound will strongly bind lithium ions such that about 90% of the repeat units will bind a lithium cation. The molar mass of this gel will be about:

0.9(290 grams/mole)+0.1(284 grams/mole)=289.4 grams/mole.

The charge density accumulated by the single valence lithium ion will be about:

p=0.9(6.022×1023) electrons/289.4 grams=300 Coulombs/gram.

The cell of the electrochemical double-layer capacitor will have a voltage of about 3.3. Volts. The energy density (E) of the gel material medium will be about:

E=pV=(300 Coulombs/gram)(3.3 Volts=990 Joules/gram=275 Watt-hours/kilogram

Accordingly, the energy density (E) of the gel material may be significantly higher than conventional power devices, such as existing capacitors (E=less than about 0.1 Watt-hours/kilogram), ultracapacitors (E=less than about 10 Watt-hours/kilogram) and batteries (E =less than about 100 Watt-hours/kilogram).

### Example 4

### Preparing a Polymer Solution of an Acrylamide-Based Conductive Compound

A solution of monomers of the acrylamide-based conductive compound 1-methyl-6-deoxy-6-(meth)acrylamide-ethyl cellulose will be formed. A crosslinking agent will be added to the solution of monomers having formula VII:

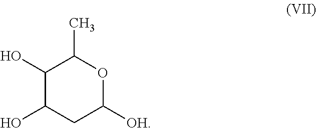

A polymer solution will be formed by adding the crosslinking agent to the solution of 1-methyl-6-deoxy-6-(meth)acrylamide-ethyl cellulose monomers. The crosslinking agent will polymerize the 1-methyl-6-deoxy-6-(meth)acrylamide-ethyl cellulose monomers through the vinyl group on the acrylamide moiety to form links having formula VIII:

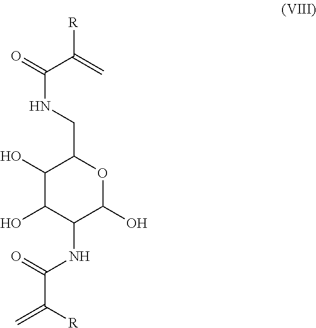

The concentration of the crosslinking agent will be configured to achieve at least 50% polymerization of the 1-methyl-6-deoxy-6-(meth)acrylamide-ethyl cellulose monomers.

### Example 5

### Preparing a Battery from an Acrylamide-Based Conducting Gel

A solution of monomers of the acrylamide-based conductive compound 1-methyl-6-deoxy-6-(meth)acrylamide-glucose will be formed. A lithium ion battery will be formed having a graphite anode and a cathode formed from Li+FePO⁴⁻ that will use a gel form of the acrylamide-based conductive compound as an electrode binding material and as the gelation material for the electrolyte.

The graphite material for the anode may be dispersed within a first volume of the acrylamide-based conductive monomer solution to form an anode slurry. The Li+FePO⁴⁻ material for the cathode may be dispersed within a second volume of the acrylamide-based conductive monomer solution to form a cathode slurry.

The graphite material for the anode may be dispersed within a first volume of the acrylamide-based conductive monomer solution to form an anode slurry. The Li+FePO⁴⁻ material for the cathode may be dispersed within a second volume of the acrylamide-based conductive monomer solution to form a cathode slurry.

Glycerol will be added to the anode slurry, the cathode slurry and an electrolyte volume of the acrylamide-based conductive compound to dissolve the acrylamide-based conductive compound monomer solution. The crosslinking agent 2-deoxy-6-deoxy-2-(meth)acrylamide-6-(meth)acrylamide-D-glucose and lithium persulfate (1% by mole) will be contacted with the anode slurry, the cathode slurry and the electrolyte volume to form an anode gel solution, a cathode gel solution and an electrolyte gel solution, respectively. The anode gel solution, the cathode gel solution and the electrolyte gel solution will be heated to about 80° C. to form a solid or semi-solid anode material, cathode material, and electrolyte material, respectively.

The anode and the cathode for the battery will be formed from the anode material and the cathode material, respectively. An electrolyte solution of LiBF₄ will be added to the electrolyte material to form an electrolyte gel for the battery. The gel materials formed from the acrylamide-based conductive compound will coordinate lithium ions to support the flow of lithium ions between the anode and the cathode to generate a voltage within the battery. The voltage is expected to be at least 0.9 V. As the acrylamide-based conductive compound is formed from organic liquids that are generally non-flammable or have high boiling points, and the metal ions are coordinated with the acrylamide-based conductive compound, runaway reactions and spontaneous decomposition of the battery due to fast diffusion of the ions is likely to be prevented or reduced.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1.-17. (canceled)

18. A polymer comprising formula II: 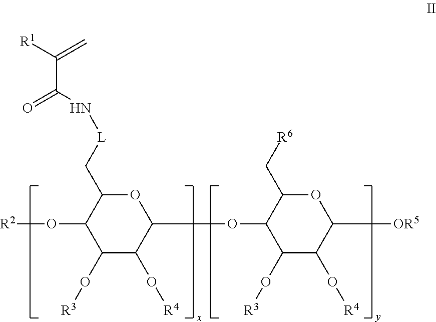
the polymer being a random polymer, a block polymer or an alternating polymer;
wherein
L is a linking moiety selected from a direct bond, —O—, —R⁷—, —R⁷—O—, —R⁷—O—C(O)—R⁷—;
R¹ is H, —CH₃, or —C₁-C₆ alkyl-OH;
R₂ is H or —CH₃;
R₃ is H or —CH₃;
R⁴ is H or —CH₃;
R⁵ is H or —CH₃;
each R⁶ is independently —C₁-C₆ alkyl-OH;
each R⁷ is independently selected from —O—, —R⁸—, —R⁸—O—, —R⁸—O—C(O)—R⁸—;
each R⁸ is independently selected from C₁-C₆ alkyl;
x is an integer of 1 to 100;
y is an integer of 0 to 100; and
z is an integer of 0 to 100.

19. The polymer of claim 18, wherein R¹, R², R³, and R⁴ are H.

20. The polymer of claim 18, wherein R¹, R², R³, and R⁴ are —CH₃.

21.-22. (canceled)

23. The polymer of claim 18, wherein R² is —CH₃, R³ is H, and R⁴ is H.

24. The polymer of claim 18, wherein R⁷ is —O—.

25. The polymer of claim 18, wherein R⁷ is —R⁸—O—.

26. The polymer of claim 18, wherein R⁷ is —R⁸—O—C(O)—R⁸—.

27. The polymer of claim 18, wherein R¹, R², R³, R⁴ and R⁵ are each individually selected from an alkyl group, an alkene group, an alkyne group, or an aryl group.

28. The polymer of claim 18, wherein L is a direct bond or —O—.

29.-34. (canceled)

35. The polymer of claim 18, coordinated with at least one metal ion of a metal selected from the group consisting of lithium, sodium, potassium, magnesium, calcium, iron, and copper.

36.-57. (canceled)

58. A method of making an acrylamide-based conductive material, the method comprising:
contacting an acetone/pyridine solution with a carbonate solution to form a first intermediate compound;
contacting 1-methyl-6-deoxy-6-ammonium bromide-D-glucose with the first intermediate compound to form a second intermediate compound;
contacting hexane with (meth) acryloyl chloride to form a third intermediate compound;
contacting the third intermediate compound with the second intermediate compound to form a fourth intermediate compound;
removing water from the fourth intermediate compound to form a solid compound;
contacting an alcohol with the solid compound to dissolve organic material in the solid compound;
removing the alcohol; and
forming the acrylamide-based conductive material.

59. The method of claim 58, wherein contacting the acetone/pyridine solution with a carbonate solution comprises contacting with carbonate solution formed by dissolving one or more of a lithium bicarbonate and a sodium bicarbonate compound in water.

60.-67. (canceled)

68. The method of claim 58, wherein forming the acrylamide-based conductive material by comprises recrystallizing the solid compound

69. The method of claim 59, wherein the one or more lithium bicarbonate and sodium bicarbonate is present in the bicarbonate carbonate solution in an amount of about 11% by weight per volume of solution.

70. (canceled)

71. The method of claim 58, wherein contacting the hexane and the (meth) acryloyl chloride comprises contacting at a volume ratio of about 1:0.03.

72. (canceled)

73. The method of claim 58, wherein further comprising forming the acrylamide-based conductive material into the acrylamide-based conductive gel by:
dissolving the acrylamide-based conductive material in a mixture comprising an organic fluid, a crosslinking agent, and a metal persulfate to form a gel solution; and
heating the gel solution to form the acrylamide-based conductive gel.

74. The method of claim 73, wherein heating the gel solution comprises heating at a temperature of about 70° C. to about 80° C.

75. (canceled)

76. The method of claim 73, wherein dissolving the acrylamide-based conductive material comprises dissolving in a the metal persulfate comprises comprising at least one of: lithium persulfate, sodium persulfate, potassium persulfate, magnesium persulfate, and calcium persulfate.

77. (canceled)

78. The method of claim 73, wherein dissolving the acrvlamide-based conductive material comprises dissolving in an organic fluid comprising at least one of the following: glycerol, sorbitol, ethylene glycol, and propylene glycol.

79.-80. (canceled)

81. An acrylamide-based conductive gel battery comprising:
an acrylamide-based conductive gel infused with electrolyte; and
at least one anode and at least one cathode arranged within the acrylamide-based conductive gel, wherein the acrylamide-based conductive gel supports ionic communication between the at least one anode and the at least one cathode, the ionic communication generating an electric current for the acrylamide-based conductive gel battery.

82.-84. (canceled)

85. The battery of claim 81, wherein the electric current generated by the ionic communication between the at least one anode and the at least one cathode yields a battery voltage of at least about 0.9 V.

86. (canceled)

87. The battery of claim 81, wherein:
the at least one cathode comprises a cathode material selected from copper, carbon, silver, metal oxides, conducting polymers, carbon, carbon nanotubes, graphite, graphene, germanium, silicon, titanate, and combinations thereof;
the at least one anode comprises an anode material selected from zinc, magnesium, aluminum, calcium, lithium, conducting polymers, iron, nickel, metal oxides, and combinations thereof; and
the electrolyte comprises at least one of: lithium hexafluorophosphate, lithium hexafluoroarsenate monohydrate, lithium perchlorate, lithium tetrafluoroborate, and lithium triflate.

88.-89. (canceled)

90. The battery of claim 81, wherein the electrolyte comprises a complex counter-ion selected from cobalt (IV) oxide, manganese oxide, nickel oxide, and iron (III) phosphate.

91. (canceled)

92. A compound having the formula IV: 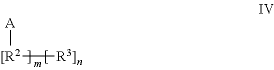
wherein A is an acrylamide moiety represented by the formula V: 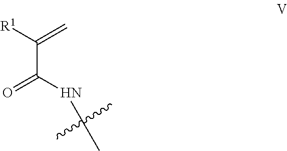
wherein
R¹ is H or —CH₃;
R² is a polysaccharide, glucose, fructose, cellulose, hydroxyethyl cellulose, cellulose acetate butyrate, ethyl cellulose, a mogroside, mogroside II A₁, mogroside II A₂, mogroside II B, mogroside V, mogroside VI, and 7-oxomogroside II E;
R₃ is a polysaccharide, glucose, fructose, cellulose, hydroxyethyl cellulose, cellulose acetate butyrate, ethyl cellulose, a mogroside, mogroside II A₁, mogroside II A₂, mogroside II B, mogroside V, mogroside VI, and 7-oxomogroside II E;
m is an integer of 1 to 100; and
n is an integer of 0 to 100.

93. The compound of claim 92, wherein R² is selected from the group consisting of glucose, fructose, and cellulose.

94.-95. (canceled)

96. The compound of claim 92, wherein R² is hydroxyethyl cellulose, cellulose acetate butyrate, or ethyl cellulose.

97. The compound of claim 92, wherein R² is a mogroside selected from the group consisting of mogroside II A₁, mogroside II A₂, mogroside II B, mogroside V, mogroside VI, and 7-oxomogroside II E.

98. (canceled)

99. The compound of claim 92, wherein R³ is selected from the group consisting of glucose, fructose and cellulose.

100.-102. (canceled)

103. The compound of claim 92, wherein R³ is a mogroside selected from the group consisting of mogroside II A¹, mogroside II A₂, mogroside IIB, mogroside V, mogroside VI, and 7-oxomogroside II E.

104.-109. (canceled)

110. The compound of claim 92, wherein ion further comprising a coordination metal ion of a metal selected from the group consisting of lithium, sodium, potassium, magnesium, calcium, iron, or copper.

111. (canceled)
